(19) United States
(12) Patent Application Publication
(10) Pub. No.: US 2024/0245612 A1
(43) Pub. Date: Jul. 25, 2024

(54) COMPOSITIONS AND METHODS FOR REDUCING ADVERSE EFFECTS OF STORAGE, TRANSPORT AND ADMINISTRATION OF ANTIGEN-CONTAINING FORMULATIONS

(71) Applicant: THE REGENTS OF THE UNIVERSITY OF COLORADO, A BODY CORPORATE, Denver, CO (US)

(72) Inventors: Theodore Randolph, Niwot, CO (US); Alyssa Witeof, Westminster, CO (US); Robert Garcea, Boulder, CO (US)

(21) Appl. No.: 18/627,155

(22) Filed: Apr. 4, 2024

Related U.S. Application Data
(63) Continuation of application No. PCT/US2022/077708, filed on Oct. 6, 2022.
(60) Provisional application No. 63/253,063, filed on Oct. 6, 2021.

(51) Int. Cl. A61K 9/50; A61K 9/48; A61K 39/00; A61K 39/385

(52) U.S. Cl. CPC A61K 9/501; A61K 9/4858; A61K 39/385; A61K 2039/54

(57) ABSTRACT

Embodiments of the instant disclosure generally relate to novel compositions, methods, and systems for reducing or eliminating effects of interfering agents or premature dissolution of antigen-containing microparticles during storage, transport, and delivery of antigen-containing formulations. Certain embodiments concern improved formulations for more reliable storage, transport, delivery, and administration of metal oxide coated antigen-containing suspension formulations.

### PRIORITY

This U.S. Non-Provisional application is a continuation of International Application No. PCT/US2022/077708, filed Oct. 6, 2022, which claims the benefit of U.S. Provisional Application No. 63/253,063, filed Oct. 6, 2021, which are all incorporated herein by reference in their entireties for all purposes.

### FIELD

Embodiments of the instant disclosure generally relate to novel compositions, methods, and systems for reducing or eliminating effects of interfering agents or premature dissolution of antigen-containing complexes during storage, transport, and delivery of antigen-containing formulations. Certain embodiments concern improved, and reliable storage, transport, and delivery of formulations including metal oxide coated antigen-containing suspensions or suspended formulations.

### BACKGROUND

There are several challenges presented by storing liquid formulations of antigen-containing agents in vials and syringes. Some formulations containing therapeutic proteins are adversely affected when exposed to hydrophobic interfaces, such as those presented by silicone oil lubricants, elastomeric stoppers, and septa. Air-water interfaces as well as container-liquid interfaces such as water-container interfaces are other sources of, or adverse effects during storage of antigen-containing formulations which can destabilize, cause reduced product delivery, and cause degradation of therapeutic products.

Other issues concern destabilization of target agents in liquid formulations that can cause destabilization or unwanted coupling of target agents due to adverse molecular interactions with antimicrobials and preservatives. Other problems arise from using dried formulations which alleviate some issues with liquid formulations but create other issues. For example, filling vials with dry powders is more challenging than filling with liquid formulations and dry powders and lyophilized formulations typically must be reconstituted with water or a buffer solution prior to administration. In addition, if preservatives and antimicrobials are added to the reconstitution medium, the stability of the antigen-containing product can be compromised due to interference of the preservatives and antimicrobials with the target agent. Therefore, needs exist to improve storage, transport, and ease of and more accurate delivery of antigen-containing agents susceptible to interference, irregularities in dispensing and degradation due in part to these identified factors.

### SUMMARY

Embodiments of the instant disclosure generally relate to novel compositions, methods, and systems for improving stability of time-release formulated antigens, stability of coated antigens, and reducing or eliminating effects of interfering agents on storage, transport and delivery of antigen-containing formulations. Some embodiments concern stabilizing metal oxide-coated antigens in the form of suspended particles or microparticles whereby suspending formulations disclosed herein maintain continuity and stability of coated antigen particles. Certain embodiments concern improved and reliable storage, transport, and delivery of coated or layered antigen-containing formulations to improve reliability in delivery of antigens of use to ameliorate, treat or prevent a health condition in a subject.

In some embodiments, coated or layered antigens disclosed herein include, but are not limited to, metal oxide-coated antigens. In accordance with these embodiments, metal oxide-coated antigens can include coated spray freeze-dried antigens in a glassy state. In certain embodiments, coated antigens can include atomic layer deposition (ALD) coated antigens (e.g., antigenic proteins, polypeptides, polypeptides, polysaccharides, polynucleotides, microorganisms (e.g., viruses, fungi, bacteria, prion), small molecules, or fragments thereof) that have undergone a drying process prior to being coated. In accordance with certain embodiments, ALD-coated antigens can be in the form of particles, such as microparticles in a dry or powdered state for improving stability of the coated antigen(s), agent compatibility, storage, and transport. In certain embodiments, metal oxide coated (e.g., ALD coated) microparticles containing one or more antigen can be suspended for delivery to a subject where a suspension buffer includes a non-chelating organic agent alone or in combination with other agents.

In some embodiments, formulations disclosed herein can include combinations of one or more ALD-coated microparticle containing the same or different antigens and further include, but are not limited to, formulations for improving microparticle integrity, reducing storage component interferences and additive interferences for improved and reliable coated antigen delivery to a subject. Embodiments disclosed herein provide formulations for prolonged storage of, and ready administration of, metal oxide coated antigen containing-microparticles for efficient and accurate delivery of target antigens to prevent, treat or reduce onset of a condition or infection.

In some embodiments, the instant disclosure relates to improved storage and delivery of controlled-release, powdered vaccine formulations previously disclosed (See for example, PCT/US2017/019163 filed Feb. 23, 2017). In certain embodiments, a combination of spray drying, and atomic layer deposition (ALD) can be used to coat antigen particles embedded in a glassy matrix for improved stabilization. In certain embodiments, a glassy matrix can be provided by any agent known in the art capable of forming a glassy matrix while in essentially dried form (e.g., trehalose, sucrose, mannitol and sucrose or similar)

In other embodiments, target antigens or antigens contemplated herein include, but are not limited to, immunogenic agents for example, polypeptides, polynucleotides including RNA, DNA, hybrid molecules, lipid coated polynucleotides, virus-like particles, viruses, bacterial derived agents, bacteriophages or agents derived therefrom, polysaccharides, chimeric polypeptides or polynucleotides, or combination polynucleotides, toxins, microorganisms or fragments thereof, small molecules, or fragments or derivatives thereof can be formulated into coating-ready particles such as glassy matrices which are stable. In accordance with these embodiments, essentially dried microparticles of coating-ready antigens can include formulations known in the art used in preparation of the coating ready particles including, but not limited to, trehalose, sucrose, mannitol and sucrose or similar agent or combination thereof. (See for example PCT/US2015/029529 filed May 6, 2015, incorporated herein by reference in its entirety).

In other embodiments, antigens contemplated herein for use in treating, reducing onset of, or preventing a health condition can be readily available for metal oxide or other suitable metal ion coating. It is contemplated herein that any metal oxide or metal ion coated antigen-containing microparticle can be in a dry state or essentially dry state ready for suspension in non-chelating organic buffers having reduced or low ionic strength contemplated herein for prolonged storage in a ready-to-use state.

In some embodiments disclosed herein, powders produced by processes disclosed herein (e.g., ALD coated microparticles containing antigens) can be suspended or resuspended in an aqueous medium having reduced interferences from storage components and/or storage agents used to preserve the composition. In accordance with these embodiments, an aqueous media can include, but is not limited to, a non-ionic tonicity modifying agent-containing buffer. In some embodiments, the buffer includes a non-chelating organic buffer. In certain embodiments, the non-chelating organic buffer includes, but is not limited to, histidine or similar non-chelating organic buffer. In other embodiments, a buffer or aqueous media cannot be an inorganic buffer capable of dissolving metal oxide coatings or that is chelating for example, a phosphate or citrate buffer or a phosphate-citrate combination buffer, a solution containing chelating agents (e.g., ethylenediaminetetraacetic acid (EDTA)) or other agent capable of dissolving or reducing the integrity of metal oxide coating. In certain embodiments, a formulation for coated microparticles disclosed herein can include histidine and/or phosphate (e.g., sodium phosphate). In accordance with these embodiments, the histidine and/or phosphate concentration can be about 40 mM or less; or about 30 mM or less; about 20 mM or less; about 10 mM or less; or about 5 mM or less. In some embodiments, the pH of the suspension formulation including histidine and/or phosphate is less than about pH 8.0 when the concentration of the histidine and/or phosphate is about 40.0 mM or less; 30 mM or less; 20 mM or less or 10 mM or less. In some embodiments, the pH of the suspension formulation including histidine and/or phosphate is less than about pH 8.0 when the concentration of the histidine and/or phosphate is about 5.0 mM or less. In some embodiments, the pH of the suspension formulation including histidine and/or phosphate is about pH 7.0 or more when the concentration of the histidine and/or phosphate is about 5.0 mM or less. In certain embodiments, the pH of the suspension formulation including histidine is about 7.0 or more; or about 7.0 to about 8.5; or about 7.5 to about 8.5 when the concentration of histidine is less than 20 mM; or 15 mM or less; or 10 mM or less; or 5 mM or less. In certain embodiments, these suspension formulations can further include one or more additional agents for stabilizing and/or facilitating delivery of the coated microparticles containing one or more antigen.

In some embodiments, suspension formulations for coated antigen-containing microparticles can include histidine and be stored at any temperature for storage, transport, and delivery to a subject. In accordance with these embodiments, suspension formulations containing histidine can be stored at less than about 4º C to about 60° C.′ or more, or to about 50° C. or more, for several hours to about a week, a month, two months or more without premature or unscheduled release of the one or more coated antigens. In some embodiments, these suspension formulations do not include phosphate. In certain embodiments, these formulations can further include one or more additional agents for stabilizing and/or facilitating delivery of the coated microparticles containing one or more antigen (e.g., trehalose). In some embodiments, suspension formulations for coated antigen-containing microparticles can include histidine and trehalose (or sucrose or similar agent) and be stored at less than about 4º C to about 60° C.′ or more, or to about 50° C. or more, for several hours to about a week, a month, two months or more without premature or unscheduled release of the one or more coated antigens enhancing storage, transport, and delivery of coated antigen-containing microparticles. In other embodiments, a suspension formulation disclosed herein does not include aqueous phosphate, citrate, or sulfate. In other embodiments, a suspension formulation disclosed herein does not include a chelating agent. In other embodiments, a suspension formulation disclosed herein does not include EDTA. In other embodiments, a suspension formulation disclosed herein can further include a salt at a concentration of less than 0.1M.

In certain embodiments, suspension formulations disclosed herein can further include trehalose or sucrose or the like. In certain embodiments, trehalose and/or sucrose concentrations can be about 20% w/v or less. In certain embodiments, trehalose and/or sucrose concentrations can be about 15% w/v or less. In yet other embodiments, suspension formulations disclosed here can include a surfactant; for example, polysorbate.

In some embodiments, suspension formulations disclosed herein do not include buffers containing, citrate, phosphate, or a salt thereof such as sodium salt. In certain embodiments, suspension formulations disclosed herein do not include buffers containing citrate. In certain embodiments, suspension formulations disclosed herein do not include buffers containing citrate-phosphate formulations. In accordance with these embodiments, citrate alone or in combination with phosphate induces instability in coated antigen microparticles disclosed herein. In other embodiments, suspension formulations disclosed herein do not include buffers containing phosphate salt buffer.

In other embodiments, a composition can include one or more surfactant or another similar agent. In other embodiments, compositions disclosed herein can include an anti-microbial agent. In yet other embodiments, suspension or an aqueous formulation of use herein can include one or more preservative (e.g. benzyl alcohol, methylparaben, creosol, phenols, etc.).

In other embodiments, with regards to the embodiments disclosed herein and at least paragraphs [0006]-[0015] above, antigens or target agents within the suspended microparticles in a non-chelating buffer having one or more layers of metal oxide coating (e.g. ALD) are thermally stable. Advantages of these formulations include, but are not limited to, that suspended particles provide a more readily-fillable format into vials, tubes, syringes, or other containers more efficiently than powders using for example, traditional liquid fill-finishing equipment. Another advantage of using an aqueous formulation is that a formulation as a stable suspension of powders or stable suspension of metal oxide or ALD-coated microparticles eliminates the need for suspension in preparation for, or at the time of administration of vaccines, immunogenic compositions or the therapeutic agent of interest. In addition, these stable formulations are in a ready-to-inject formulation of suspended powders or microparticles eliminating the need for a second vial of suspension liquid, decreasing shipping weight and improving convenience for caregivers. In yet other embodiments, metal oxide or ALD-coated microparticles create layers protecting the antigen such as a polypeptide or a polynucleotide or small molecule embedded within the ALD-coated microparticle powder from damage by an antimicrobial agent or other preservative, extending the shelf life of single and multidose, preservative-containing formulations. In yet other embodiments, antigens coated with atomic layer deposition (ALD) and suspended in an aqueous medium are protected or insulated from adverse interactions with container surfaces, container closure elements (e.g., stoppers, septa, and syringe plungers), lubricants (e.g., silicone oils) and other interfaces.

Other embodiments disclosed herein concern kits for storage, transport and use of suspended metal oxide coated antigens disclosed herein. In certain embodiments, a kit can include a ready-to-use syringe containing a suspension formulation disclosed herein for delivering a dose of a target antigen to a subject. Other embodiments concern multidose formulations with improved stability and reliability.

The following drawings are intended to be examples and are not to be construed in any way as limiting.

### BRIEF DESCRIPTION OF THE DRAWINGS

FIG. 1 illustrates zeta potential of metal oxide coated microparticles in varying buffers and pH of certain embodiments disclosed herein.

FIGS. 2A-2B represent exemplary graphs illustrating pre-mature leakage of antigen from coated particles in the presence and absence of various buffers at room temperature and an elevated temperature, in the presence or absence of a surfactant; (2A) by IR detection or by SDS-PAGE and (2B) by IR with release into the suspension buffer of certain embodiments disclosed herein.

FIG. 3 represents a plot demonstrating release of antigen from coated particles in the presence or absence of various buffers under different temperature conditions and analyzed by IR and SDS-PAGE of certain embodiments disclosed herein.

FIG. 4 represents a plot demonstrating release of antigen from coated particles in the presence or absence of various buffers under different temperature conditions over a time course of incubation and analyzed by IR and SDS-PAGE of certain embodiments disclosed hercin.

FIG. 5 illustrates plots of spray dried microparticles of exemplary antigens embedded in a glassy matrix coated with a metal oxide by atomic layer deposition (ALD). The coated microparticles are then suspended in aqueous buffers and release of antigen from the microparticles into the buffers is measured in certain embodiments disclosed herein.

FIG. 6 illustrates a graph of percent release of antigen of coated microparticles suspended in a suspension buffer containing a chelating agent over several days of incubation in certain embodiments disclosed hercin.

FIG. 7 represents an exemplary schematic of metal oxide coats applied by ALD to a microparticle containing at least one antigen in certain embodiments disclosed herein.

FIG. 8 illustrates an exemplary process represented by a schematic demonstrating metal oxide coating of antigens in certain embodiments disclosed herein.

FIGS. 9A-9C illustrates schematic diagrams of an antigen being coated by metal oxide layering (9A); a second different antigen added to an outside layer of a partially coated particle (9B); and a second coating of the same or different antigen (9C) of certain embodiments disclosed herein.

### DEFINITIONS

Terms, unless specifically defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that may vary from about 10% to about 15% plus and/or minus depending upon the desired properties sought as disclosed herein. Numerical values as represented herein inherently contain standard deviations that necessarily result from the errors found in the numerical value's testing measurements.

As used herein, the term “subject” can refer to any mammal, including but not limited to, a non-human primate (for example, a monkey or great ape), livestock or pets such as a cow, a pig, a cat, a dog, a rat, a mouse, a horse, a goat, a rabbit, a sheep, a hamster, a guinea pig) or other subject. In some embodiments, the mammalian subject is a human such as an adult, a young child, adolescent, toddler, infant or fetus.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details may be modified through routine experimentation. In some cases, well known methods, or components have not been included in the description.

Embodiments of the instant disclosure generally relate to novel compositions, methods, and systems for improving stability of suspension of time-release formulated antigens, stability of coated antigens, reducing or eliminating effects of interfering agents on storage and delivery of antigen-containing formulations. Some embodiments concern stabilizing metal oxide-coated antigens in the form of dry particles once suspended whereby formulations disclosed herein maintain stability of coated antigen particles. Certain embodiments concern improved and reliable storage, transport, and delivery of coated antigen-containing formulations.

In some embodiments, coated antigens disclosed herein include, but are not limited to, metal oxide coated antigens, for example, coated spray freeze-dried antigens in a glassy state. In accordance with these embodiments, coated antigens can include atomic layer deposition (ALD) coated antigens (e.g., proteins, polypeptides, polynucleotides, microorganisms, small molecules, or fragments thereof) that have undergone a drying process or thermal stable transition prior to being coated. In accordance with certain embodiments, ALD-coated antigens can be in the form of particles, such as microparticles in an essentially dry or powdered state for improving stability of the coated antigen(s), agent compatibility, storage, and transport. In certain embodiments, metal oxide coated (e.g., atomic layer deposition (ALD) coated) microparticles containing one or more antigen can be suspended for delivery to a subject where a suspension buffer includes, a non-chelating organic agent at reduced ionic strength.

In some embodiments, formulations disclosed herein can include combinations of one or more ALD-coated microparticle containing the same or different antigens and further include, but are not limited to, formulations for improving microparticle integrity (e.g., reducing or eliminating pre-mature antigen release) reducing storage component interferences and additive interferences for improved and reliable antigen delivery to a subject. Embodiments disclosed herein provide formulations for prolonged storage of, and ready administration of suspended metal oxide-coated antigen containing-microparticles for efficient and accurate delivery of target antigens to treat or reduce onset of a condition or treat or reduce onset of an infection or spread of an infection.

In some embodiments, the instant disclosure relates to improved storage and delivery of controlled-release, powdered vaccine formulations previously disclosed (See for example, PCT/US2017/019163 filed Feb. 23, 2017, incorporated herein by reference for all purposes). In these methods, a combination of spray drying, and atomic layer deposition (ALD) can be used to coat antigen particles embedded in a glassy matrix. In other embodiments, target antigens including, but not limited to, polypeptides, polynucleotides including RNA, DNA, lipid coated polynucleotides or combination polynucleotides, microorganisms, small molecules, virus-like particles, viruses, polysaccharides, or fragments or derivatives thereof can be formulated into coating-ready particles such as glassy matrices which are stable. In accordance with these embodiments, essentially dried microparticles of coating-ready antigens can include formulations known in the art in preparation of the coating ready particles including, but not limited to, trehalose, sucrose or combination thereof, (See for example PCT/US2015/029529 filed May 6, 2015 incorporated herein by reference in its entirety for all purposes). In some embodiments, antigen-containing formulations can be lyophilized in the presence of glass-forming excipients, and sufficient liquid can be removed during lyophilization that the dried or essentially dried formulation or immunogenic composition exhibits a glassy stable state ready for metal oxide or other suitable metal ion coating. In other embodiments, antigens contemplated herein for use in treating, reducing onset of, or preventing a condition can be readily available for metal oxide or other suitable metal ion coating. It is contemplated herein that any metal oxide or metal ion coated antigen-containing microparticle can be in a dry state ready for suspension in non-chelating organic buffers contemplated herein for prolonged storage in a ready-to-use state.

In some embodiments disclosed herein, powders produced by processes disclosed herein (e.g., ALD coated microparticles containing antigens) can be suspended or resuspended in an aqueous medium or suspension formulation having reduced interferences or adverse effects from storage components and/or storage agents use to preserve the composition. In accordance with these embodiments, an aqueous media can include, but is not limited to, a non-ionic tonicity modifying agent-containing buffer. In some embodiments, the buffer includes a non-chelating organic buffer. In certain embodiments, the non-chelating organic buffer or agent includes, but is not limited to, histidine, imidazole, glycine, bis tris methane, tris, bicine, glycylglycine or similar non-chelating organic buffer or other agent or buffer referred to a Good's or amine buffer known by those of skill in the art. In other embodiments, buffers of use in suspension formulations disclosed herein can include, but are not limited to, MES, Bis-tris methane, ADA, Bis-tris propane, PIPES, ACES, MOPSO, cholamine chloride, MOPS, BES, TES, HEPES, DIPSO, MOBS, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, CAPSO, AMP, CAPS, CABS or the like. These buffers do not include sulfate-containing agents. In other embodiments, a buffer or aqueous media cannot include an inorganic buffer capable of dissolving metal oxide coatings or that is chelating for example, a phosphate or citrate buffer or a phosphate-citrate combination buffer or other agent capable of dissolving or reducing the integrity of metal oxide coating. In certain embodiments, phosphate, citrate, or similar chelating buffers are avoided or the concentration is reduced to avoid dissolving metal oxide coatings of microparticles disclosed herein and to reduce pre-mature release of antigens from metal-oxide coated particles or microparticles.

In other embodiments, concentrations of organic buffers can be at a reduced or minimal ionic strength to; for example, reduce interferences that can occur with the coating of a metal-oxide coated target antigen or active agent such as coating degradation or pre-mature coating dissolution. In certain embodiments, organic buffers for particle suspension contemplated herein reduce premature antigen release to improve accuracy and predictability of dosing upon delivery to a subject. In accordance with these embodiments, an inorganic or organic buffer can be about 100.0 mM or less, or about 50.0 mM or less, or about 25.0 mM or less or about 10.0 mM or less or about 5.0 mM or less or about 1.0 mM or less. In other embodiments, a composition can include one or more surfactant such as a high molecular weight surfactant or other agent. In certain embodiments, a surfactant can include a non-ionic surfactant including, but not limited to, polysorbate 80, polysorbate 20, or poloxamer 188 or poloxamer 403 or poloxamer 407, Tween 20, Tween 80, or the like. In other embodiments, compositions disclosed herein can include an anti-microbial agent. In yet other embodiments, suspension or aqueous media of use herein can include one or more preservative (e.g., benzyl alcohol, methylparaben, parabens, chlorobutanol, phenol, sorbic acid, cresol, metacresol, or other known preservative). In accordance with these embodiments, a resulting suspension is stable, while reducing or preventing release of antigen into the suspending medium for about a day, about a week, about two weeks, several weeks, about a month, about 6 weeks, about two months, about 10 weeks, about three months, about 6 months or more.

In other embodiments, with regards to the embodiments disclosed herein, antigens or target agents within the suspended particles in a non-chelating buffer having one or more layers of metal oxide coating (e.g., ALD) are thermally stable for about a day, about a week, about two weeks, several weeks, about a month, about 6 weeks, about two months, about 10 weeks, about three months, about 6 months or more at temperatures of less than 4° C. up to about 50° C. to about 55° C. or about 60° C.

Some advantages of the formulations disclosed herein, include, but are not limited to, that suspended particles provide a more readily fillable format into vials, tubes, syringes, bottles, droppers, atomizers, or other containers than powders using traditional liquid fill-finishing equipment. Another advantage is that formulation as a stable suspension of powders or stable suspension of metal oxide or ALD-coated microparticles eliminates the need for suspension in preparation for, or at the time of administration of vaccines. In addition, these stable formulations are in a ready-to inject formulation of suspended powders eliminating the need for a second vial of suspension liquid, decreasing shipping weight and improving convenience for caregivers. In yet other embodiments, metal oxide or ALD-coated microparticles create layers protecting the antigen such as a polypeptide or polynucleotide or small molecule embedded within the ALD-coated microparticle powder from damage by an antimicrobial agent or other preservative, extending the shelf life of single and multidose, preservative-containing formulations. In yet other embodiments, antigens coated with ALD and suspended in an aqueous medium are protected or insulated from adverse interactions with surfaces and other interfaces. In accordance with these embodiments, suspended ALD-coated microparticles protect antigens (e.g., polypeptide, polynucleotide, microorganism, small molecules, or fragments thereof) from incompatibilities with container materials and closure systems. In some embodiments, suspended ALD-coated microparticles have reduced incompatibility with syringe lubricants (e.g., silicone oil), interfaces presented by air bubbles, and surfaces present in glass or polymeric containers, and polymeric surfaces such as vial stoppers and syringe plungers. In other embodiments, suspended ALD-coated microparticles disclosed herein can be suspended in a buffer that is isotonic making them suitable for immediate parenteral administration to a subject at the point of care. In other embodiments, suspended ALD-coated microparticles of suspensions disclosed herein can be stable, liquid formulations that reduce or eliminate cold-chain requirements for target antigen or vaccine storage and transportation. In accordance with these embodiments, formulations for suspending metal oxide coated antigen particles can include a histidine buffer, a histidine-like buffer or other, non-chelating buffer. In certain embodiments, the pH of the non-chelating buffer (e.g., histidine) can be about 4.0 to about 8.0, or about 5.0 to about 7.5 or other suitable pH.

In certain embodiments, a suspension formulation for coated microparticles disclosed herein can include histidine, imidazole, glycine, bis tris methane, tris, bicine, glycylglycine or similar non-chelating organic buffer or other agent or buffer referred to a Good's or amine buffer known by those of skill in the art. In certain embodiments, concentrations of buffers or agents used as suspension formulations disclosed herein can be reduced (e.g., 20 mM or less) and can further include phosphate (e.g., sodium or potassium phosphate). In accordance with these embodiments, the histidine and/or phosphate concentration can be about 40 mM or less; or about 30 mM or less; about 20 mM or less; about 10 mM or less; or about 5 mM or less. In some embodiments, a suspension formulation for coated microparticles disclosed herein can include sodium phosphate at a concentration of about 5 mM or less. In other embodiments, a suspension formulation for coated microparticles disclosed herein can include histidine at a concentration of about 5 mM or less. In accordance with these embodiments, the pH of the suspension formulation including histidine and/or phosphate is less than about pH 8.0 when the concentration of the histidine and/or phosphate is about 20.0 mM or less. In some embodiments, the pH of the suspension formulation including histidine and/or phosphate is less than about pH 8.0 when the concentration of the histidine and/or phosphate is about 15.0 mM or less. In some embodiments, the pH of the suspension formulation including histidine and/or phosphate is less than about pH 8.0 when the concentration of the histidine and/or phosphate is about 10.0 mM or less. In some embodiments, the pH of the suspension formulation including histidine and/or phosphate is less than about pH 8.0 when the concentration of the histidine and/or phosphate is about 5.0 mM or less. In some embodiments, the pH of the suspension formulation including histidine and/or phosphate is about pH 7.0 or more when the concentration of the histidine and/or phosphate is about 20.0 mM or less. In some embodiments, the pH of the suspension formulation including histidine and/or phosphate is about pH 7.0 or more when the concentration of the histidine and/or phosphate is about 15.0 mM or less. In other embodiments, the pH of the suspension formulation including histidine and/or phosphate is about pH 7.0 or more when the concentration of the histidine and/or phosphate is about 10.0 mM or less. In some embodiments, the pH of the suspension formulation including histidine and/or phosphate is about pH 7.0 or more when the concentration of the histidine and/or phosphate is about 5.0 mM or less. In certain embodiments, the pH of the suspension formulation including phosphate is about 6.0 to about 8.0; or about 6.0 to about 7.5 when the concentration of phosphate is about 20.0 mM or less; or 15 mM or less; or 10 mM or less; or 5 mM or less. In certain embodiments, the pH of the suspension formulation including histidine is about 7.0 or more; or about 7.0 to about 8.5; or about 7.5 to about 8.5 when the concentration of histidine is less than 20 mM; or 15 mM or less; or 10 mM or less; or 5 mM or less. In certain embodiments, these suspension formulations can further include one or more additional agents for stabilizing and/or facilitating delivery of the coated microparticles containing one or more antigen.

In some embodiments, suspension formulations for coated antigen-containing microparticles can include histidine and be stored at any temperature for storage, transport, and delivery to a subject. In accordance with these embodiments, suspension formulations containing histidine can be stored at less than about 4º C to about 60° C.′ or more, or to about 50° C. or more, for several hours to about a week, a month, two months or more without premature or unscheduled release of the one or more coated antigens. In some embodiments, these suspension formulations do not include phosphate. In certain embodiments, these suspension formulations can further include one or more additional agents for stabilizing and/or facilitating delivery of the coated microparticles containing one or more antigen (e.g., trehalose). In some embodiments, suspension formulations for coated antigen-containing microparticles can include histidine and trehalose (or sucrose or similar agent) and be stored at less than about 4º C to about 60° C.′ or more, or to about 50° C. or more, for several hours to about a week, a month, two months or more without premature or unscheduled release of the one or more coated antigens enhancing storage, transport, and delivery of coated antigen-containing microparticles. In certain embodiments, a formulation including histidine does not include phosphate. In other embodiments, a suspension formulation disclosed herein does not include aqueous phosphate, citrate, or sulfate. In other embodiments, a suspension formulation disclosed herein can further include a salt at a concentration of less than about 0.200 M, or about 0.150 M or about 0.100 M or less.

In certain embodiments, suspension formulations disclosed herein can further include trehalose or sucrose or other agents. In accordance with these embodiment, a disaccharide agent such as trehalose or sucrose or other comparable disaccharide or sugar can be used to achieve isotonicity of the composition. In certain embodiments, trehalose and/or sucrose concentrations can be about 20% w/v or less. In certain embodiments, trehalose and/or sucrose concentrations can be about 15% w/v or less. In other embodiments, suspension formulations can further include one or more salt (e.g., sodium salt, potassium salt). In certain embodiments, multivalent anion agents can be present at a concentration of about 50 mM or less while amine-based agents (e.g., histamine) or buffers can be present at a concentration of about 100 mM or less in suspension formulations as indicated herein. In yet other embodiments, suspension formulations disclosed here can include a surfactant; for example, polysorbate.

In some embodiments, suspension formulations disclosed herein do not include buffers containing, citrate, phosphate, or a salt thereof such as sodium. In certain embodiments, suspension formulations disclosed herein do not include buffers containing citrate. In certain embodiments, suspension formulations disclosed herein do not include buffers containing citrate-phosphate formulations. In accordance with these embodiments, citrate alone or in combination with phosphate induces instability in coated antigen microparticles disclosed herein. In other embodiments, suspension formulations disclosed herein do not include buffers containing phosphate salt buffer. In certain embodiments, suspension formulations disclosed herein do not include buffers containing sodium-phosphate formulations. In accordance with these embodiments, buffers containing citrates, phosphates, or chlorides were particularly effective at causing particles to adversely change. Suspension and administration of coated antigen microparticles disclosed herein require selection of the diluent for injection to avoid stability problems in the final product. In certain embodiments, suspension formulations disclosed herein should avoid multivalent anions (e.g., citrate, phosphate, sulfate) to maintain microparticle integrity.

In other embodiments, a composition can include one or more surfactants such as a non-ionic surfactant (e.g., polysorbate 20, polysorbate 80, poloxamer 188), or another similar agent. In other embodiments, compositions disclosed herein can include an anti-microbial or preservative agent (e.g., benzyl alcohol, methylparaben, creosol, phenols, etc.). In other embodiments, if preservatives and antimicrobials are added to the formulation used to suspend metal oxide-coated antigen microparticles, target antigens protected by metal oxide coating can be further protected from adverse reactions to additives by suspending the metal oxide-coated antigens in a non-chelating buffer of low ionic strength disclosed herein. In accordance with these embodiments, surfactants, antimicrobials, and preservatives can be added to these formulations without damaging antigens embedded within the metal oxide-coated microparticles. In certain embodiments, suspension formulations or compositions disclosed herein include non-chelating buffers at low ionic strength and further include one or more of a preservative or a surfactant or other suitable storage agent. In accordance with these embodiments, a resulting suspension is stable, while reducing or preventing release of antigen from the coated microparticles into the suspending formulation before delivery to a subject.

Formulation of vaccines and therapeutic proteins in dried forms (e.g., created by lyophilization or spray drying) can alleviate some of the potential damage to vaccines and protein therapeutics caused by exposure to various interfaces (e.g., silicone oil-water interfaces, air-water interfaces, container-water interfaces), preservatives and antimicrobials. In some embodiments, compositions and methods disclosed herein concern embedding or encapsulation of vaccines or therapeutic proteins within a dry powder matrix or within a lyophilized formulation cake to reduce or prevent direct contact with container stoppers, seals, plungers, and lubricants. In accordance with these embodiments, some interferences are alleviated but dried forms of formulations can pose additional challenges. In other embodiments, filling vials with dry powders is more challenging than filling with liquid formulations and dry powders and lyophilized formulations must still be suspended in a medium or formulation prior to injection. Embodiments disclosed herein take advantage of the stable nature of metal oxide coated antigen microparticles at the same time as formulating these microparticles within a liquid or aqueous suspension for ready distribution, storage, transport, and use.

In some embodiments, antigens disclosed herein having metal oxide coatings can be generated by a thermostabilization step that produces microparticles of the antigen and at least one adjuvant embedded in a glassy sugar matrix. In accordance with these embodiments, atomic layer deposition can be used to coat the glassy particle to make stable microparticles with defined atomic layers of metal oxides that dissolve over specific time periods, upon which a priming layer of antigen and potentially one or more boost layer are included. In certain embodiments disclosed herein, a non-chelating buffer can be used to suspend the microparticles for later use without jeopardizing the integrity of the microparticles and reduce pre-mature release of antigen.

Certain embodiments of the present disclosure provide methods for making the immunogenic agent-containing particles of use in suspended formulations disclosed herein, the method can include combining at least one immunogenic agent with at least one glass-forming agent to form a primary liquid immunogenic composition, dehydrating the primary liquid immunogenic composition to form immunogenic agent-containing glassy microparticles, and coating the immunogenic agent-containing glassy microparticles with one or more outer coating layers. In some embodiments, the primary liquid immunogenic composition can be dehydrated by lyophilization, vacuum-drying, spray drying, or spray-freeze-drying.

In accordance with these embodiments, the at least one immunogenic agent can include one or more antigens, for example a viral antigen, a bacterial antigen, a toxin, or a combination thereof. In some embodiments, the at least one immunogenic agent can also include but is not limited to, a recombinant peptide, a recombinant protein, a peptide derived from a target protein or pathogen, a synthetic peptide or protein, a virus-like particle, a live virus, a live, attenuated virus, an inactivated virus, or a combination thereof.

In certain embodiments, the at least one immunogenic agent capable of being coated in a formulation with metal oxide can include one or more antigens for example, antigens derived from human papilloma virus, ricin toxin, Bacillus anthracis, Clostridium botulinum, Ebola virus, poliovirus, norovirus, rotavirus, hepatitis C, varicella, herpes simplex, cytomegalovirus, Japanese encephalitis, dengue virus, West Nile virus, Zika virus, Yersinia, Pneumococcus, Salmonella, Clostridium difficile, or a combination thereof. In certain embodiments, the at least one immunogenic agent can be a multimeric complex.

In certain embodiments, the pathogenic virus may be, for example, a papovavirus (e.g., papillomaviruses, including human papilloma virus (HPV)), a herpesvirus (e.g., herpes simplex virus, varicella-zoster virus, bovine herpesvirus-1, cytomegalovirus), a poxvirus (e.g., smallpox virus), a reovirus (e.g., rotavirus), a parvovirus (e.g., parvovirus B19, canine parvovirus), a picornavirus (e.g., poliovirus, hepatitis A), a togavirus (e.g., rubella virus, alphaviruses such as chikungunya virus), a hepadnavirus (e.g., hepatitis B virus), a flavivirus (e.g., dengue virus, hepatitis C virus, West Nile virus, yellow fever virus, Zika virus, Japanese encephalitis virus), an orthomyxovirus (e.g., influenza A virus, influenza B virus, influenza C virus), a paramyxovirus (e.g., measles virus, mumps virus, respiratory syncytial virus, canine distemper virus, parainfluenza viruses), a rhabdovirus (e.g., rabies virus), a filovirus (e.g., Ebola virus), SARS, or a coronavirus (e.g., COVID 19, delta, mu or other strain) or combinations thereof or polypeptide thereof or polynucleotide thereof as provided herein.

In other embodiments, the pathogenic agent can be a bacterium or a toxin of a bacterium, including but not limited to, Pasteurella haemolytica, Clostridium difficile, Clostridium haemolyticum, Clostridium tetani, Corynebacterium diphtheria, Neorickettsia resticii, Streptococcus equi equi, Streptococcus pneumoniae, Salmonella spp., Chlamydia trachomatis, Bacillus anthracis, Yersinia spp., and Clostridium botulinum or combinations thereof.

In some embodiments, the pathogenic agent can be a fungus, including but not limited to Cryptococcus spp. (e.g., neoformans and gatti), Aspergillus spp. (e.g., fumigatus), Blastomyces spp. (e.g., dermatitidis), Candida albicans, Paracoccidioides spp. (e.g., brasiliensis), Sporothrix spp. (e.g., schenkii and brasiliensis), Histoplasma capsulatum, Pneumocystis jirovecii and Coccidioides immitis, or combinations thereof.

In yet other embodiments, an antigen can include a pathogenic agent such as a toxin, such as ricin toxin or botulinum toxin or anthrax toxin or another toxin.

In some embodiments, immunogenic agent-containing particles described herein can be used to manufacture one or more immunogenic composition of use as vaccines for animals such as household pets. In accordance with these embodiments, the immunogenic composition can be administered, for example, to a dog (canine), a cat (feline), a horse (equine), cattle (bovine), a goat (hircine), a sheep (caprine), or poultry (e.g., chicken, turkey, duck, goose).

In certain embodiments, immunogenic agent-containing particles described herein can be used to generate one or more immunogenic compositions for administering to a canine to reduce onset of or prevent an infection, including but not limited to, infections related to canine parvovirus (CPV), canine distemper virus (CDV), canine adenovirus (CAV), rabies, canine parainfluenza virus (CPiV), canine influenza virus, canine corona virus, measles virus, Bordetella bronchiseptica, Leptospira spp., and Borrelia burgdorferi or combinations thereof.

In some embodiments, immunogenic agent-containing particles described herein can be used to generate immunogenic compositions of use for administering to a feline to reduce or prevent an infection or treat an infection, including but not limited to, immunogenic compositions directed to feline herpesvirus 1 (FHV1), feline calicivirus (FCV), feline panleukopenia virus (FPV), rabies, feline leukemia virus (FeLV), feline immunodeficiency virus, virulent systemic feline calicivirus, Chlamydophila felis, Pasteurella haemolytica, and Bordetella bronchiseptica or combinations thereof.

In other embodiments, immunogenic agent-containing particles described herein can be used to generate immunogenic compositions of use for administering to equine, to reduce or prevent an infection or treat an infection, including but not limited to, immunogenic compositions directed to Eastern equine encephalomyelitis virus, Western equine encephalomyelitis virus, Venezuelan equine encephalomyelitis virus, bovine papillomavirus, rabies virus, Clostridium tetani, West Nile virus, equine influenza virus, Potomac fever (Neorickettsia risticii), Streptococcus equi equi, and rhinopneumonitis (equine herpesvirus type 1) or combinations thereof.

In certain embodiments, immunogenic agent-containing particles described herein can be used to generate immunogenic compositions of use for administering to bovine, to reduce or prevent an infection or treat an infection, including but not limited to, immunogenic compositions directed to bovine rhinotracheitis (IBR), parainfluenza type 3 (PI3), bovine virus diarrhea (BVD), bovine respiratory syncytial virus (BRSV), blackleg (Clostridium chauvoci), malignant edema (Clostridium septicum), infectious necrotic hepatitis (Clostridium novyi), enterotoxemia (Clostridium perfringens type C and D), Pasteurella haemolytica, and redwater (Clostridium haemolyticum) or combinations thereof.

In some embodiments, immunogenic agent-containing particles described herein can be used to generate immunogenic compositions of use for administering to poultry, to reduce or prevent an infection or treat an infection, including but not limited to, immunogenic compositions directed to Marek's disease (Marek's disease virus), tenosynovitis (reoviruses), encephalomyelitis (avian encephalomyelitis virus), fowlpox (avipoxviruses), chicken infectious anemia (chicken anemia virus), fowl cholera (Pasteurella multocida), Newcastle/infectious bronchitis (Newcastle disease virus), Riemerella anatipestifer, duck viral hepatitis (duck hepatitis virus), and duck viral enteritis (duck herpesvirus 1) or combinations thereof.

In some embodiments, immunogenic agent-containing particles described herein can be used to generate immunogenic compositions of use for administering to a human. In certain embodiments, immunogenic agent-containing particles described herein may be used to deliver one or more immunogenic compositions to a human infant or child or adolescent, including but not limited to vaccines for varicella-zoster (chicken pox), diphtheria, Haemophilus influenzae type b (Hib), hepatitis A, hepatitis B, influenza, measles, mumps, pertussis, polio, pneumococcal disease, rotavirus, rubella, and tetanus. In other embodiments, immunogenic agent-containing particles described herein may be used to deliver one or more immunogenic compositions to a human pre-teen or teen, including but not limited to vaccines for influenza, tetanus, diphtheria, pertussis, human papillomavirus, meningococcal disease, hepatitis B, hepatitis A, polio, measles, mumps, rubella, and varicella-zoster. In yet other embodiments, immunogenic agent-containing particles described herein may be used to deliver one or more immunogenic compositions to a human adult, including but not limited to immunogenic compositions against influenza (e.g. A, B or C), tetanus, diphtheria, pertussis, zoster, pneumococcal disease, meningococcal disease, measles, mumps, rubella, varicella, hepatitis A, hepatitis B, and Haemophilus influenzae type b.

In other embodiments, immunogenic agent-containing particles described herein may be used to generate immunogenic compositions of use for administering to a human, including but not limited to, immunogenic compositions against travel-related diseases, including but not limited to hepatitis A, hepatitis B, typhoid fever, paratyphoid fever, meningococcal disease, yellow fever, dengue fever, rabies, Chikungunya disease, and Japanese encephalitis.

In yet other embodiments, immunogenic agent-containing particles described herein may be used to generate immunogenic compositions of use for administering to a human, including but not limited to, immunogenic compositions against human papillomavirus (e.g. HPV 16, HPV18, HPV31, HPV45, or HPV 6 or HPV11, or any other HPV), herpes simplex virus, smallpox virus, rotavirus, parvovirus B19 vaccine, chikungunya virus, dengue virus (e.g. dengue-1, dengue-2, dengue-3 or dengue-4), hepatitis C virus, West Nile virus, Zika virus, respiratory syncytial virus, rabies virus, and Ebola virus.

In some embodiments, the at least one glass-forming agent can include at least one of trehalose, sucrose, ficoll, dextran, sucrose, maltotriose, lactose, mannitol, hydroxyethyl starch, glycine, cyclodextrin, povidone, or the like. In certain embodiments, the at least one glass-forming agent can include trehalose, sucrose or hydroxyethyl starch. In certain embodiments, the at least one glass-forming agent can include trehalose. In accordance with this embodiment, trehalose can be included as a glass-forming agent and can be present in the primary liquid immunogenic composition in a weight-to-volume (w/v) concentration of from about 0.1% to about 40%, from about 1% to about 30%, from about 5% to about 20%, or from about 8% to about 15%.

In other embodiments, the glass-forming agent also includes at least one smoothing excipient. In accordance with these embodiments, the smoothing excipient can be a hydroxyethyl starch or other pharmacologically acceptable plasma expander such as human serum albumin (HSA), other serum albumins, dextran, hetastarch, plasma protein factor and the like, or a combination thereof. In certain embodiments, the smoothing excipient can also be the primary glass-forming agent. In some embodiments, the smoothing excipient is hydroxyethyl starch. In some embodiments, a smoothing agent disclosed herein can be the primary glass-forming agent. In accordance with these embodiments, the smoothing excipient can be present in the primary immunogenic composition at a weight-to-volume (w/v) concentration from about 0.1% to about 40%, from about 1% to about 30%, from about 5% to about 20%, or from about 8% to about 15%. In certain embodiments, the smoothing excipient can be different than the primary glass-forming agent, and the smoothing excipient can be present in the primary immunogenic composition in a weight-to-volume (w/v) concentration from about 0.1% to about 10%, from about 0.1% to about 5%, from about 0.1% to about 2.5%, from about 0.1% to about 0.5%. In certain embodiments, the glass-forming agent present in the immunogenic composition is trehalose and the smoothing excipient for the particles is hydroxyethyl starch.

In some embodiments, each layer of the one or more outer metal oxide coating layers can include aluminum oxide, an aluminum alkoxide (e.g., alucone), silicon dioxide (SiO2), titanium dioxide (TiO2), silicon nitride (Si3N4), zinc oxide (ZnO), zircone (MLD), zirconia, hafnium oxide, alone or in a suitable combination composition or other suitable (e.g. biocompatible) metal oxide coating. In accordance with these embodiments, the outer coating layer(s) can be about 0.1 nm to about 20 nm in thickness. In certain embodiments, the immunogenic agent-containing particle can include a number of outer coating layers sufficient to delay release or provide a timed-release of the at least one immunogenic agent from the central or innermost immunogenic agent-containing glassy microparticle.

In certain embodiments, the one or more coating layer(s) disclosed herein can serve as an adjuvant to enhance the immune response in a subject against one or more immunogenic agent(s) of the immunogenic agent-containing particle. In certain embodiments, the one or more coating layer(s) can contain a concentration capable of inducing a rapid immune response to the one or more immunogenic agent(s) of the immunogenic agent-containing particle.

In other embodiments, the immunogenic agent-containing particle can further include at least a second immunogenic agent deposited as a layer on an outermost coating layer of the immunogenic agent-containing particle. In accordance with these embodiments, the layer of the at least the second immunogenic agent can be embedded in a glassy matrix of at least a second glass-forming agent to stabilize the second immunogenic agent.

In other embodiments, methods for producing at least one layer of the at least a second immunogenic agent and embedding the second immunogenic agent in a glassy matrix of at least a second glass forming agent are also provided. In some embodiments, at least one additional outer coating layer covers or encases the layer of the at least second immunogenic agent. In certain embodiments, the second immunogenic agent is identical to the at least one immunogenic agent of the central immunogenic agent-containing glassy microparticle. In other embodiments, the second immunogenic agent is different than the at least one immunogenic agent of the central immunogenic agent-containing glassy microparticle.

In some embodiments, suspensions containing immunogenic agent-containing particles described herein can be stored without refrigeration at temperatures of up to about 50° C. to about 60º C for extended periods of time. In certain embodiments, immunogenic agent-containing particles described herein can be stored without refrigeration up to about 50° C. to about 60° C. up to about 3 months, or up to about 4 months, or up to about 6 month, or up to about 9 months, or up to about 12 months, or up to about 15 months, or up to about 18 months, or up to about 24 months or longer without negative effect on the immunogenic agent-containing particles (e.g. degradation)

Other embodiments of the present disclosure provide for immunogenic compositions including a plurality of immunogenic agent-containing particles described herein. In certain embodiments, the immunogenic composition can include immunogenic agent-containing particles in a pharmaceutically acceptable excipient to make a pharmaceutically acceptable immunogenic composition. In accordance with these embodiments, immunogenic compositions described can elicit an immune response to the immunogenic agent when administered to a subject.

In some embodiments, the immunogenic composition is a single-administration immunogenic composition comprising a prime dose and boost dose of at least one immunogenic agent. In accordance with these embodiments, the prime and boost doses of the at least one immunogenic agent can be in the same immunogenic agent-containing particle, or in separate immunogenic agent-containing particles. When in separate particles, the priming dose of the at least one immunogenic agent can be sequestered in an immunogenic agent-containing glassy microparticle without any outer coating layers, or in an immunogenic agent-containing particle, while the boost dose of the at least one immunogenic agent is in another immunogenic agent-containing particle. Whether the priming dose is in an immunogenic agent-containing glassy microparticle or in an immunogenic agent-containing particle will be determined by whether an immediate or delayed response is desired, where outer coating layers of an immunogenic agent-containing particle sequestering the prime dose will temporally delay release of the priming dose, and therefore, delay exposure of the priming dose to the subject.

In some embodiments, the immunogenic composition can be a single administration immunogenic composition capable of eliciting an immune response to two or more different immunogenic agents. In accordance with these embodiments, the two or more different immunogenic agents can be included in the same immunogenic agent-containing particles, or in separate immunogenic agent-containing particles. In other embodiments, when the two or more different immunogenic agents are contained in separate immunogenic agent-containing particles, each different immunogenic agent-containing particle comprises a different immunogenic agent or combinations of immunogenic agents.

In some embodiments, an immunogenic composition can include prime and boost doses of a first immunogenic agent and prime and boost doses of a second immunogenic agent. In accordance with these embodiments, the prime and boost doses of the first immunogenic agent are in a first immunogenic agent containing particle, while the prime and boost doses of the second immunogenic agent are sequestered in a second immunogenic agent-containing particle. In other embodiments, the prime and boost doses of the first immunogenic agent are located in a first pair of particles, the prime dose being in a separate particle from the boost dose, and the prime and boost doses of the second immunogenic agent are sequestered in a second pair of particles, the prime dose being in a separate particle from the boost dose, where the prime dose for each of the first and second immunogenic agent are in separate immunogenic agent-containing glassy microparticles and the boost dose for each the first and second immunogenic agent are in separate immunogenic agent-containing particles. In yet other embodiments, the prime and boost doses of the first immunogenic agent are sequestered in a first pair of immunogenic agent-containing particles, the prime dose being in a separate particle from the boost dose, and the prime and boost doses of the second immunogenic agent are located in a second pair of immunogenic agent-containing particles, the prime dose being in a separate particle from the boost dose, wherein the prime dose for the first immunogenic agent is in an immunogenic agent-containing glassy microparticle, the prime dose for the second antigen is in a separate immunogenic agent-containing particle, and the boost dose for each the first and second antigen are in separate immunogenic agent-containing particles.

In other embodiments, an immunogenic composition can include a standard vaccine composition and a plurality of immunogenic agent-containing particles described, wherein the at least one immunogenic agent elicits a boost immune response to the standard vaccine composition.

In yet other embodiments, an immunogenic composition can include a plurality of first immunogenic agent-containing particles described herein, wherein the first immunogenic agent-containing particles can include at least a first immunogenic agent, a plurality of second immunogenic agent-containing particles described herein, wherein the second immunogenic agent-containing particles includes at least a second immunogenic agent different than the first immunogenic agent, and a pharmaceutically acceptable excipient. In accordance with embodiments, the immunogenic composition can further include a plurality of at least one additional immunogenic agent-containing particles described herein, wherein the at least one additional immunogenic agent-containing particles include at least one additional immunogenic agent that is not the first immunogenic agent or the second immunogenic agent.

Other embodiments provide for methods for eliciting an immune response in a subject, where the method can include administering an immunogenic composition described herein to the subject. In accordance with these embodiments, the immunogenic composition can induce an immune response in the subject. The immune response induced by the immunogenic composition can be prophylactic or therapeutic depending on the immunogenic agent.

Other embodiments disclosed herein concern kits for storage, transport and use of suspended metal oxide coated antigens disclosed herein. In certain embodiments, a kit can include a ready-to-use syringe containing a suspension formulation disclosed herein for delivering a dose of a target antigen to a subject. In other embodiments, kits can include multidose container harboring suspension formulations disclosed herein for prolonged storage, reduce cold storage requirements, reliable dosing and reduced interference with storage components like stoppers, oils and preservatives. In accordance with these embodiments, a single vial of multiple doses containing a desired antigen of use to treat, reduce onset of, or prevent a health condition can be used to treat multiple subjects including human subjects and animals such as livestock.

In some embodiments, a formulation disclosed herein can include metal oxide coated antigen microparticles, a histidine buffer at a pH of about 6.0 to about 8.0, and optionally, one or more preservative and one or more surfactant agent. In accordance with these embodiments, the histidine buffer can be about 1 mM to about 50 mM.

The pharmaceutical formulations suitable for injection include sterile aqueous solutions and dispersions. The carrier can be a solvent or dispersing medium containing, for example, water, saline, polyol (for example, glycerol, propylene glycol, liquid polyethylene glycol, and the like) and suitable mixtures thereof.

In some embodiments, certain additives which enhance the stability, sterility, and isotonicity of the compositions, including antimicrobial preservatives, antioxidants, and buffers, can be added to formulations disclosed herein for improved stability and to reduce contamination. In some embodiments, antibacterial and antifungal agents can be added to reduce bacterial contamination in suspensions for parenteral administration or other mode of administration, for example, benzyl alcohol, methylparaben, paraben, chlorobutanol, phenol, sorbic acid, cresol, metacresol, and the like.

Sterile injectable solutions can be prepared by incorporating the suspensions utilized in practicing the present disclosure in the required amount of the appropriate solvent with certain amounts of the other ingredients, as desired. Such compositions can be in admixture with a suitable carrier, diluent, or excipient such as sterile water, physiological saline, glucose, dextrose, or the like.

Compositions of the present invention can be provided as liquid preparations, e.g., isotonic aqueous solutions, suspensions, emulsions, or viscous compositions, which can be buffered to a selected pH. The choice of suitable carriers and other additives can depend on the route of administration and the nature of the particular dosage form, e.g., liquid dosage form (e.g., whether the composition is to be formulated into a solution, a suspension, gel or another liquid form, such as a time release form or liquid-filled form). Solutions, suspensions, and gels normally contain a major amount of water (e.g., purified, sterilized water) in addition to the suspended particles or suspended microparticles. Minor amounts of other ingredients such as pH adjusters (e.g., a base such as NaOH), emulsifiers or dispersing agents, buffering agents, preservatives, wetting agents, and jelling agents (e.g., methylcellulose), can also be present. The compositions can be isotonic, i.e., they can have the same osmotic pressure as blood and lacrimal fluid.

In some embodiments, desired isotonicity of the compositions of the present disclosure can be accomplished using sodium chloride, or other pharmaceutically acceptable agents such as dextrose, boric acid, sodium tartrate, propylene glycol, or organic solutes. Viscosity of the compositions, if desired, can be maintained at the selected level using a pharmaceutically acceptable thickening agent. Methylcellulose is readily and economically available and is casy to work with. Other suitable thickening agents include, for example, xanthan gum, carboxymethyl cellulose, hydroxypropyl cellulose, carbomer, and the like. The concentration of the thickener will depend upon the agent selected. The point is to use an amount, which will achieve the selected viscosity. Viscous compositions are normally prepared from solutions by the addition of such thickening agents.

Compositions can be administered in dosages and by techniques well known to those skilled in the medical and veterinary arts taking into consideration such factors as the age, sex, weight, and condition of the particular patient, and the composition used for administration (e.g., liquid). Dosages for humans or other mammals can be determined without undue experimentation by the skilled artisan, from this disclosure, and the knowledge in the art.

In some embodiments, kits are contemplated of use to store and transport compositions disclosed herein either in the dry state or a suspended state and include at least one container. In certain embodiments, kits can include at least one composition of metal-oxide coated microparticles of a target antigen in dry form, and a buffer containing a non-chelating buffer for suspension of the microparticles. In other embodiments, kits can include ready-to-use suspended metal-oxide coated microparticles of at least one target antigen (or a combination of target antigens) in single or multi-dose container such as a vial or syringe. In certain embodiments, a non-chelating buffer includes histidine.

In some embodiments, the kits are vaccine kits of ready-to-use suspended metal-oxide coated microparticles of a target antigen of use to reduce onset or prevent infection by a microbial agent such as a virus. In certain embodiments, the antigen of any formulation disclosed herein can include, but are not limited to, a polypeptide (e.g. recombinant, chimera or naturally occurring), polynucleotide (RNA, DNA or a hybrid molecule), small molecule, a microbial agent such as a virus or bacteria, a fungus, a prion, a toxin, or other antigen.

In some embodiments, the kit can include instructions for use in accordance with any of the methods described herein. Instructions found in a kit can include a description of administration of the formulation, and optionally, a second therapeutic agent, to treat, delay the onset, or alleviate a target condition as those described herein. The kit can further include a description of selecting an individual suitable for treatment based on identifying whether that individual has or is suspected of developing the target condition. In yet other embodiments, the instructions can include a description for administering a pre-filled ready-to-administer syringe of a vaccine formulation disclosed herein to a subject at risk of developing a disease or condition disclosed herein.

In some embodiments, instructions relating to the use of a ready-to-use antigen-containing formulation generally include information including, but not limited to, dosage and treatment schedules. Containers of kits can include unit dosing or bulk packages (e.g., multi-dose packages) or sub-unit doses. Kits can further include a delivery device such as a syringe, implant device or other timed-delivery device. Instructions supplied in the kits of the invention are typically written instructions on a label or package insert (e.g., a paper sheet included in the kit), but machine-readable instructions (e.g., instructions carried on a magnetic or optical storage disk) are also acceptable.

In some embodiments, kits can be in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit can have a sterile access port (for example the container can be an intravenous solution bag or a vial having a stopper pierceable by a hypodermic injection needle). The container can also have a sterile access port (for example the container can be an intravenous solution bag or a vial having a stopper pierceable by a hypodermic injection needle). Kits contemplated herein can contain at least one active agent in the composition such as one or more antigen coated in metal oxide or ALD coated as described herein.

Kits can optionally provide additional components such as buffers and interpretive information. Normally, the kit includes a container and a label or package insert(s) on or associated with the container. In some embodiments, the invention provides articles of manufacture comprising contents of the kits described above.

### EXAMPLES

The following examples are included to illustrate certain embodiments and are not considered limiting to the instant disclosure. It should be appreciated by those of skill in the art that the techniques disclosed in the examples which follow represent techniques discovered to function in the practice of the claimed methods, compositions and apparatus. However, those of skill in the art should, in light of the present disclosure, appreciate that changes can be made in some embodiments and examples which are disclosed and still obtain a like or similar result without departing from the spirit and scope of the invention.

### Example 1

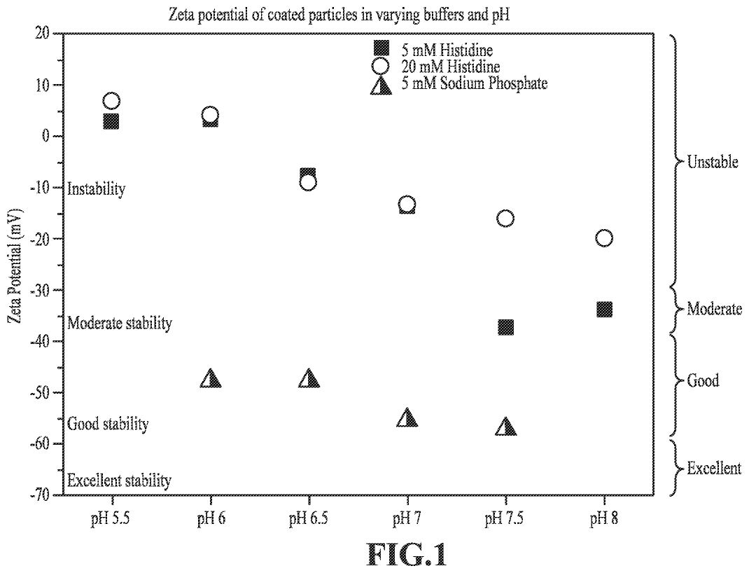

In one exemplary method, colloidal stability of atomic layer deposition-coated particles in inorganic (e.g., phosphate) and organic (e.g., histidine) buffers were investigated for stability and integrity of suspended microparticles. Spray dried particles in a formulation of trehalose, histidine, ammonium acetate and polysorbate 20 were coated (e.g., 250) molecular layers of alumina in an atomic layer deposition reactor. The resulting particles were suspended in buffers containing about 9.5% trehalose and either 5 mM histidine, 20 mM histidine, or 5 mM sodium phosphate with varying pH. Particles were suspended to a concentration of about 1.0 mg/ml. Zeta potential was evaluated. Zeta potential is a measure of colloidal stability often used to predict the interactions within therapeutic formulations. A formulation is considered unstable if the zeta potential falls between 20 and −30 mV. Moderate stability is indicated by values between −30 and −40 mV. Good stability is indicated by stability between −40 and −60 mV. Excellent stability is indicated by values less than −60 mV. Particles in 20 mM histidine were colloidally unstable at all pH values tested, whereas particles suspended in histidine buffer at minimal concentration such as 5 mM Histidine were only unstable at pH 7 and below. In contrast, 5 mM sodium phosphate exhibited good stability from pH 6 to 7.5. (See for example, FIG. 1)

### Example 2

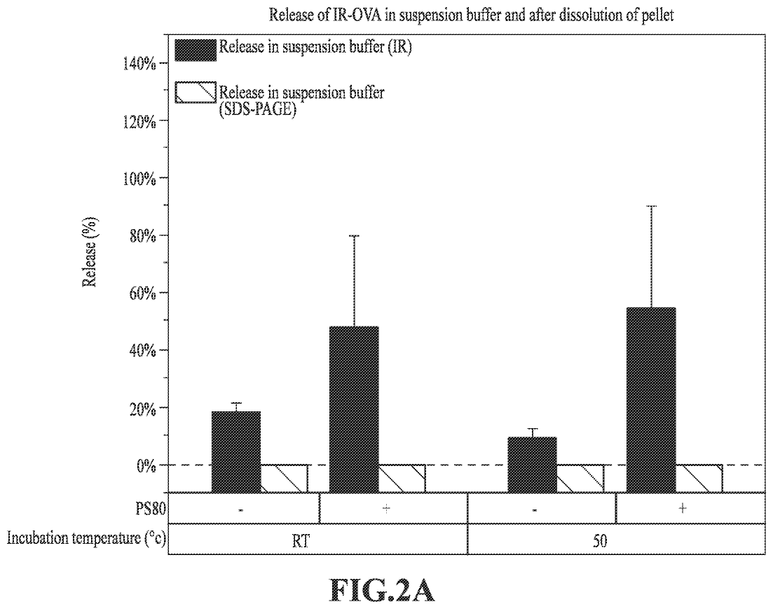

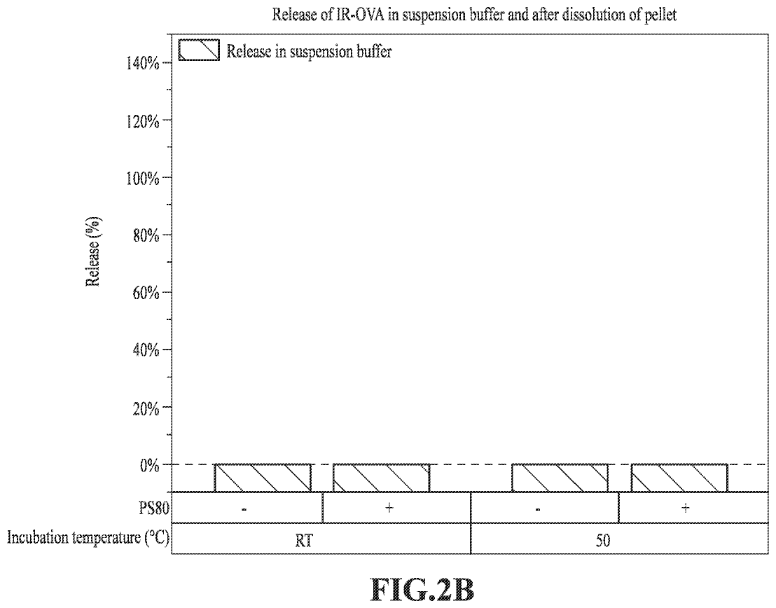

In another exemplary method, addition of polysorbate 80 to suspending media does not significantly alter the release profile, maintaining integrity of the antigen-containing particles. In this method, spray dried particles including an exemplary antigen (e.g., ovalbumin) labeled with a dye that fluoresces in the infrared (IR-OVA), trehalose, histidine, ammonium acetate and polysorbate 20 were coated with molecular layers of alumina in an atomic layer deposition reactor. Coated particles were suspended in about 9.5% trehalose, 5 mM histidine, 150 mM sodium chloride buffer with or without 0.04% polysorbate 80 and incubated for 1 week at either room temperature or 50° C. After incubation, the samples were centrifuged for 5 minutes at 16,000×g and the supernatant decanted. The remaining pellet was dissolved in a citrate-phosphate buffer to remove the alumina coating and evaluate the remaining protein. The dissolved pellets were analyzed for IR-Ovalbumin by measuring the absorbance at 672 nm. Additionally, the decanted suspension buffer was analyzed on an SDS-PAGE gel and any bands quantified. Minimal release of IR-OVA from the particles into the suspension media was observed via SDS-PAGE gels. Addition of polysorbate 80 did not significantly alter the release of the protein. (See FIGS. 2A and 2B, for example)

FIG. 2A represents an analysis of IR-dye labelled ovalbumin released from ALD-coated powders. Particles were first suspended in 9.5% trehalose, 5 mM histidine, 150 mM sodium chloride for one week. Light gray bars represent the amount of protein released into the suspending medium after one week of incubation. Dark bars represent protein released from the particles after particles were collected by centrifugation and ALD coatings were removed by dissolution in a citrate-phosphate buffer. Light gray bars represent the amount of protein released into the suspending medium after one week of incubation.

FIG. 2B represents an exemplary plot of release of IR labeled ovalbumin from ALD-coated microparticles after incubation at room temperature or 50° C. for one week in 9.5% trehalose, 5 mM histidine, 150 mM sodium chloride, in the presence and absence of 0.05% polysorbate 80.

### Example 3

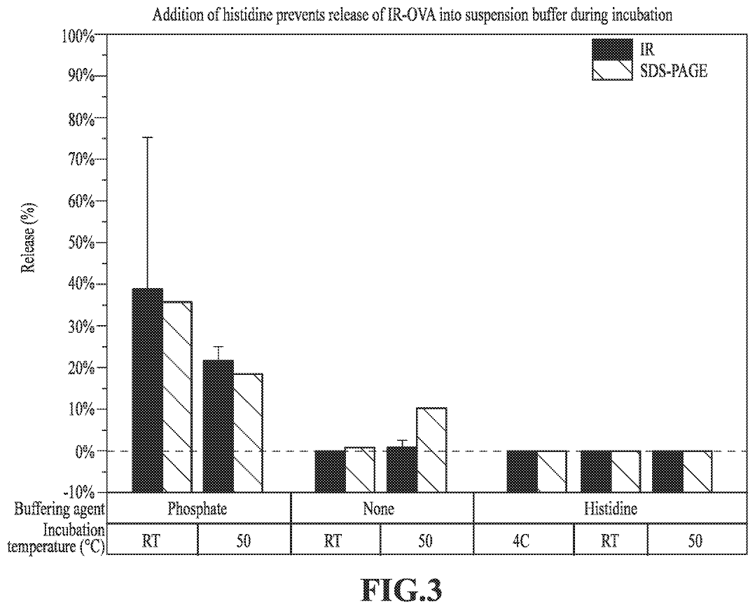

In another example, spray dried particles including an exemplary antigen (e.g. ovalbumin) labeled with a dye that fluoresces in the infrared (IR-OVA), trehalose, histidine, ammonium acetate and polysorbate 20 were coated with molecular layers of a metal ion (e.g., alumina) in an atomic layer deposition reactor. Coated particles were suspended in about 9.5% trehalose with 5 mM sodium phosphate, 5 mM histidine, or no buffering agent and incubated for 1 week at room temperature or 50° C. After incubation, the samples were centrifuged for 5 minutes at 16,000×g and the suspension buffer removed. The suspension buffer and dissolved pellet were analyzed for IR-Ovalbumin release by measuring the absorbance at 672 nm. Additionally, the suspension buffer was analyzed on an SDS-PAGE gel and any bands quantified. The inclusion of phosphate as a resuspension buffer resulted in release of protein into the suspension buffer during the incubation period. In samples lacking a buffering agent (no histidine or phosphate) there were minimal levels of protein that released. In contrast, samples that contained histidine at lower concentrations had no detectable protein release in the suspension buffer regardless of incubation temperature. These experiments support use of a non-chelating organic buffer for long term maintenance of integrity of antigen containing metal oxide antigens in ready-to-use or easily hydratable formulations. In addition, these non-chelating organic buffer formulations are projected to reduce interferences caused by preservatives such as anti-microbials, oils, silicones, waxes, or other component used to secure closure of a container used in storage of therapeutic agents and for therapeutic delivery devices providing a ready-to-use single or multidose formulation for use in a single day or over the course of weeks and even months (e.g. in remote areas for humans or for pet or livestock use to treat more than one subject using the same vial). (See for example, FIG. 3)

FIG. 3 represents a plot of release of IR labeled ovalbumin after incubation at room temperature or 50° C. for one week after suspension in 9.5% trehalose solutions containing phosphate, histidine, or no buffering agent.

### Example 4

In another exemplary experiment, coated particles containing an IR labeled ovalbumin (IR-OVA, an example antigen) were suspended in buffers containing either 9.5% trehalose, 5 mM histidine or 9.5% trehalose, 5 mM phosphate and incubated for up to 8 weeks at either room temperature or 50° C. After incubation, the samples were centrifuged for 5 minutes at 16,000×g and the suspension buffer removed. The remaining pellet was dissolved in a citrate-phosphate buffer to remove the alumina coating and evaluate the remaining protein. The suspension buffer was analyzed for IR-Ovalbumin by measuring the absorbance at 672 nm. Samples containing phosphate in the suspension buffer released IR-OVA from the particles into the suspension buffer at all time points and all temperatures. In contrast, samples containing histidine showed undetectable release regardless of incubation time or temperature. It was observed that trehalose-histidine suspension buffers prevent release into the suspension buffer of antigen for up to 8 weeks of incubation in the histidine containing formulation.

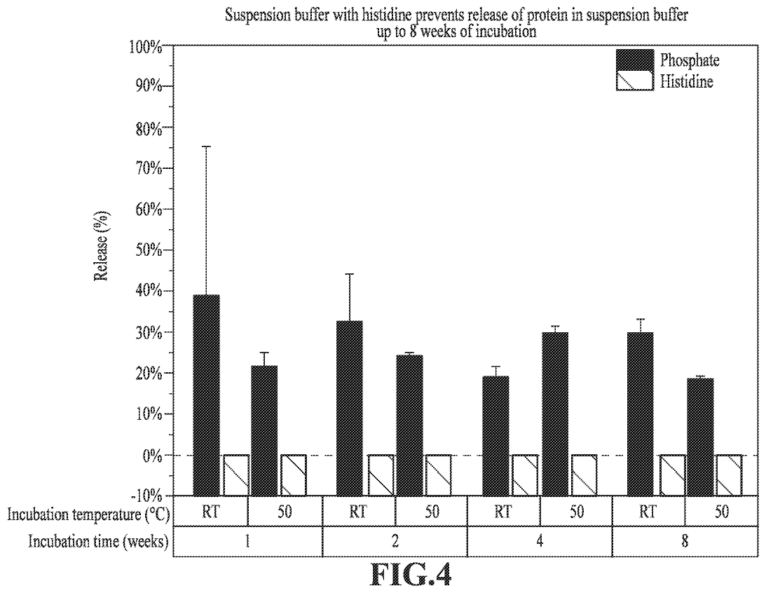

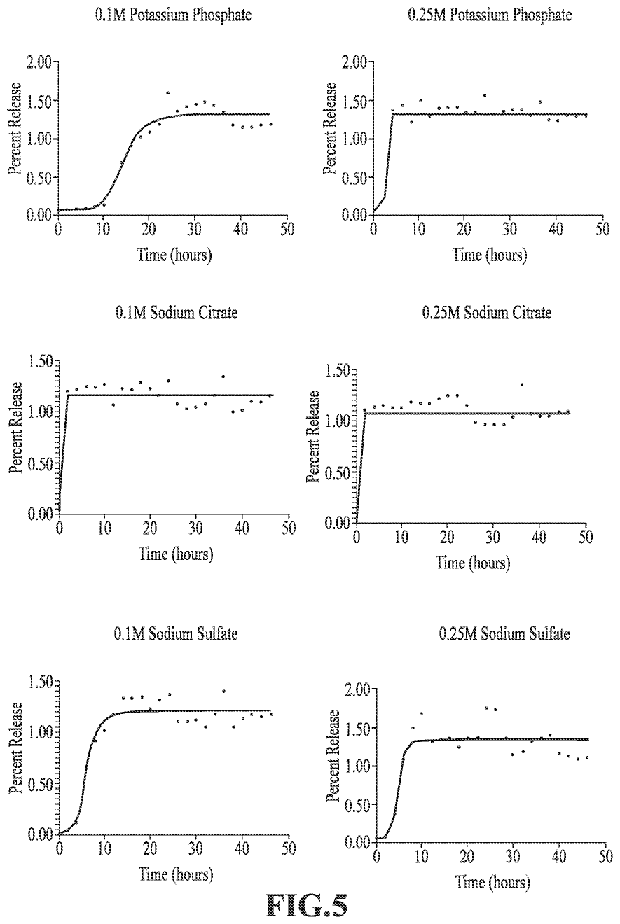

FIG. 4 represents a plot of release of IR labeled ovalbumin after incubation at room temperature or 50° C. for one, two, four, or eight weeks. Coated particles were suspended in a trehalose-histidine or trehalose-phosphate buffer prior to incubation.

### Example 5

In another exemplary experiment, spray dried microparticles including a protein antigen embedded in a glassy disaccharide matrix were coated with 100 layers of metal oxide (e.g., alumina) by atomic layer deposition (ALD). It was observed that when suspended in aqueous phosphate, citrate, or sulfate solutions, the ALD-coated microparticles rapidly release protein from the microparticles into the suspension medium. The data points for each buffer illustrate the fraction of the protein originally contained within the microparticles that has been released into the suspension medium at each time point, whereas the line represents an empirical fit to a dissolution model. Protein concentration was measured using an o-phthaladehyde fluorescent assay (OPA). Release of protein from the microparticles is more rapid in solutions containing sulfate and citrate than in solutions containing potassium phosphate. For each of suspensions tested, release of protein from the microparticles was faster at higher concentration of salt. This is a counter-example to the claimed invention regarding for example using histidine-containing formulations. This data demonstrates that ALD-coated microparticles are unstable in solutions of commonly-used, multivalent salts. In comparison, see FIG. 2B described herein, where addition of histidine prevented release of protein from microparticles.

### Example 6

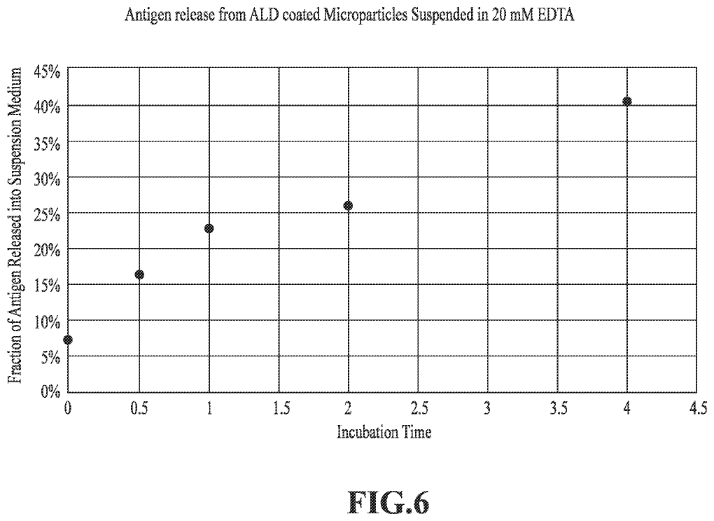

In another exemplary method, powders containing a protein antigen labeled with an IR-active dye and embedded in a glassy trehalose-based matrix were prepared by spray-drying. The powders were then coated with 500 coats of alumina using ALD. Coated powders were suspended in an aqueous solution containing a chelating agent (e.g., 20 mM EDTA), and release of the IR-dye-labeled protein antigen from the microparticles was monitored over 4 days using infrared spectroscopy. (See FIG. 6, for example)

FIG. 6 illustrates a graph of percent release of antigen from coated microparticles suspended in a suspension buffer containing a chelating agent over several days of incubation in certain embodiments disclosed herein.

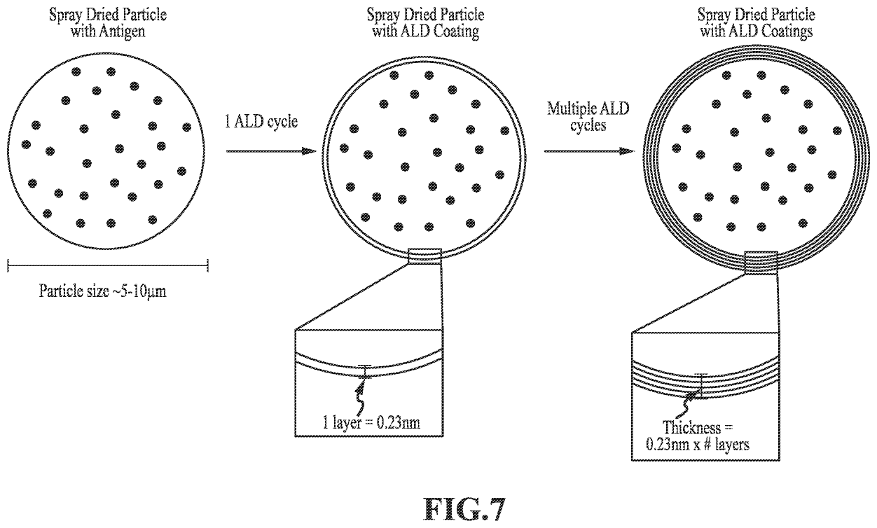

FIG. 7 represents an exemplary image of coating essentially dried microparticle containing at least one antigen. In this example, spray drying produces a thermostable particle containing APIs (antigens and adjuvants). Atomic Layer Deposition (ALD) applies a covalently attached molecule-thick layer of metal oxide to surface of particles ALD coating (Al₂O₃) is insoluble in water and dissolves slowly in vivo. Multiple ALD cycles allow coating thickness to be varied as desired. This layering enables extended or delayed release of antigen/adjuvant to a subject delivered such formulation.

FIG. 8 illustrates an exemplary process represented by a schematic demonstrating metal oxide coating of antigens illustrating a first and second coating of the antigen using metal oxide layering by ALD processes.

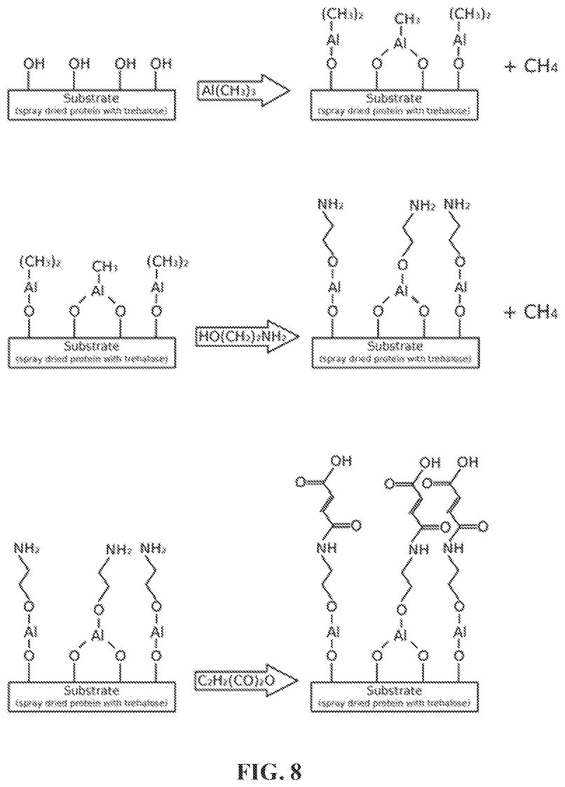

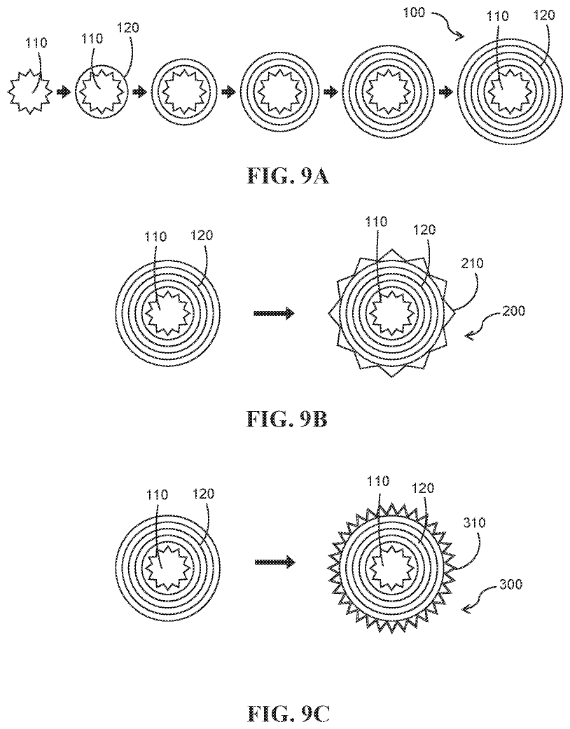

FIGS. 9A-9C illustrates schematic diagrams of an antigen being coated by metal oxide layering (9A); a second different antigen added to an outside layer of a partially coated particle (9B); and a second coating of the same or different antigen (9C) of certain embodiments disclosed herein. These diagrams illustrate priming, prime-boost and multi-antigen dosing using coated microparticles described herein.

What is claimed is:

1. An aqueous composition comprising:
microparticles containing one or more antigens coated in at least one layer of metal-oxide; and a formulation comprising one or more non-chelating agent, wherein the formulation is an aqueous formulation.

2. The composition according to claim 1, wherein the one or more non-chelating agent comprises one or more of histidine, imidazole, glycine, bis tris methane, tris, bicine, glycylglycine or similar non-chelating organic buffer.

3. The composition according to claim 1, wherein the one or more non-chelating agent comprises at least one of histidine and imidazole.

4. The composition according to any one of claims 1-3, wherein the non-chelating agent concentration is about 0.1 mM to about 100.0 mM.

5. The composition according to claim 1, wherein the coated one or more antigens comprise atomic layer deposition (ALD) coated one or more antigens comprising coated antigen-containing microparticles.

6. The composition according to claim 5, wherein the ALD coating comprises at least one covalently attached molecule-thick layer of the metal oxide over the one or more antigens.

7. The composition according to any one of claims 1 to 6, further comprising one or more of a preservative, a surfactant or antimicrobial.

8. The composition according to claim 7, wherein the one or more preservative comprises at least one of benzyl alcohol, methylparaben, paraben, chlorobutanol, phenol, sorbic acid, cresol, metacresol, and other preservatives.

9. The composition according to claim 1, wherein the composition does not contain a chelating agent.

10. The composition according to any one of claims 1-9, wherein the composition does not contain at least one of sulfate and citrate.

11. The composition according to any one of claims 1-10, wherein the one or more antigens of the metal-coated one or more antigens comprise one or more of polypeptides, polynucleotides, hybrid molecules of polypeptides and polynucleotides, hybrid molecules of polynucleotides, microorganisms, viruses, virus-like particles, bacteria, bacteriophage, fungi, polysaccharides, toxins, or fragments thereof or small molecules.

12. The composition according to claim 1, wherein the buffer comprising the non-chelating agent is isotonic.

13. The composition according to any of the preceding claims wherein the pH of the composition is about pH 6.0 to about pH 8.0.

14. The composition according to claim 1, wherein the microparticles containing one or more antigens coated in at least one layer of metal-oxide were essentially dry immediately prior to combining with the aqueous formulation.

15. A method for increasing stability of suspended antigen-containing metal oxide-coated microparticles comprising introducing an aqueous formulation to essentially dried antigen-containing metal oxide coated microparticles wherein the aqueous formulation comprises one or more non-chelating agent; and reducing release of antigens from the antigen-containing metal oxide coated microparticles.

16. The method according to claim 15, wherein the one or more non-chelating agent comprises one or more of histidine, imidazole, glycine, bis tris methane, tris, bicine, glycylglycine or similar non-chelating organic buffer.

17. The method according to claim 15 or 16, wherein the aqueous formulation further comprises one or more preservative.

18. The method according to any one of claims 15-17, wherein the concentration of the non-chelating agent in the aqueous formulation comprises 0.1 mM to 100 mM.

19. The method according to any one of claims 15-18, wherein the pH of the aqueous formulation comprises a pH of about 6.0 to about 8.0.

20. The method according to any one of claims 15-19, wherein the antigen-containing metal oxide-coated microparticles comprise antigen-containing atomic layer deposition (ALD) coated microparticles.

21. The method according to claim 15, wherein the one or more antigens comprise one or more polypeptides, polynucleotides, hybrid molecules of polypeptides and polynucleotides, hybrid molecules of polynucleotides, microorganisms, viruses, virus-like particles, bacteria, bacteriophage, fungi, polysaccharides, toxins, or fragments thereof or small molecules.

22. A kit comprising the aqueous composition according to any one of claims 1-14, and at least one container.

23. The kit according to claim 22, wherein aqueous composition comprises a multidose formulation.

24. The kit according to claim 22 or 23, wherein the aqueous composition is contained in one or more ready-to-administer delivery devices.

25. The kit according to claim 24, wherein the one or more ready-to-administer delivery devices comprises one or more syringes.
